(19) United States
(12) Patent Application Publication
(10) Pub. No.: US 2005/0070489 A1
(43) Pub. Date: Mar. 31, 2005

(54) Use of dsrnas in strategic therapeutic intervention of highly active antiretroviral therapy

(76) Inventors: William A Carter, Spring City, PA (US); David R Strayer, Bryn Mawr, PA (US)

Correspondence Address: NIXON & VANDERHYE, PC, 1100 N GLEBE ROAD, 8TH FLOOR, ARLINGTON, VA, 22201-4714, US

(21) Appl. No.: 10/500,613

(22) PCT Filed: Dec. 13, 2002
(86) PCT No.: PCT/US02/39890

(30) Foreign Application Priority Data
Dec. 14, 2001 (US) 60/339390

(51) Int. Cl. A61K 48/00; A61K 31/551; A61K 31/522

(52) U.S. Cl. 514/44; 514/220; 514/263.38

(57) ABSTRACT

In the treatment of HIV administration of dsRNA at an appropriate stage in highly active antiretroviral (HAART) therapy of HIV allows for the discontinuation of HAART by increasing the time to HIV rebound after stopping HAART.

### BACKGROUND OF THE INVENTION

Sixteen antiviral agents are currently approved by the FDA for the treatment of HIV infection. All target the specific HIV enzymes, reverse transcriptase (RT) or protease. The use of various combinations of these drugs is referred to as highly active anti-retroviral therapy (HAART) and has provided dramatic decreases in morbidity and mortality of HIV infection. Reduction of the plasma HIV RNA to undetectable levels in patients with wildtype virus (i.e. non-RT or protease resistant) is routinely possible with the appropriate application of HAART. Reduction of EIV loads potentially enables reconstitution of the immune system and led to early speculation that HIV could be eliminated by HAART. Subsequent experience has provided a more realistic view of HAART and the realization that chronic HIV suppression using HAART, as currently practiced, would require treatment for life with its resultant significant cumulative toxicities. Moreover, chronic HAART results in loss of HIV-specific cytotoxic T-lymphocytes (CTL) and memory responses.

Chronic therapy with HAART is necessitated by integration of the HIV DNA provirus in CD4+ resting memory cells that are not targets of HAART until activation of replicating HIV. Because of the long half-life of these cells, current estimates suggest that it would require as many as 60 years of HAART for elimination of HIV in the infected patient. Cumulative toxicities from HAART are currently a major contributor to non-compliance and non-acceptance for such long-term treatment requirements. Moreover, non-compliance by patients results in sub-optimum levels of HAART drugs which facilitates the development of RT and protease resistant HIV mutants. Although more potent second generation drugs are under development that target the RT and protease genes as well as new HIV targets, the problem of drug toxicities, the complex interactions between these drug classes, and the likelihood of life-long therapy will remain a serious drawback to their usage. The recent concept and limited experience with Strategic Therapeutic Interruption (STI) of HAART provides a unique opportunity to minimize the current deficiencies of HAART while retaining the superb HIV suppression capacities of HAART.

STI is the cessation of HAART for a prescribed period of time during which HIV again becomes detectable (i.e. rebound) followed by resumption of HAART with subsequent suppression of HIV. During HAART suppression of HIV, the immune system becomes desensitized to HIV antigens presented by HLA I molecules. By allowing a transient rebound of HIV during the STI of HAART the immune system may become sensitized to the patient's own virus. By reinstitution of HAART, HIV is suppressed before it can inflict damage to the immune system

TABLE 1
HAART-Based Toxicities

| | | Principle | | |
|---|---|---|---|---|
| | | Serum | | |
| | | Laboratory | Principle | |
| Metabolic | HAART | Markers of | Cell | |
| Abnormality | Components | Toxicity | Toxicity | Phenotypic Effect |
| Lipid Storage | PI^{a} | Cholesterol, | Adipocytes | Lipodystrophy^{d,e} |
| | | triglycerides | | (peripheral fat wasting |
| | | | | abdominal/dorsal |
| | | | | cervical accumulation) |
| Glucose | PI | C-peptide, | Liver, | Altered glucose |
| Utilization | | insulin, glucose^{c} | muscle | metabolism insulin- |
| | | | | resistant diabetes^{e} |
| Mitochondrial | NRTI/NNRTI^{b} | Lactic acid | Variable | Pancreatitis, |
| Function | | | | neuropathy, myopathy, |
| | | | | nephritis, osteopenia |
| Hepatic | PI/NRTI | ALT, HBs | Hepatocytes | Severe liver toxicity |
| Membrane | | antigen, | | |
| Integrity | | HCV RNA | | |

^{a}Protease inhibitors
^{b}Nucleoside and non-nucleoside reverse transcriptase inhibitors
^{c}Oral glucose tolerance test
^{d}All PIs and some NRTIs induce lipodystrophy
^{e}With chronic use there is potential for significant adverse effects on the cardiovascular system (ie; coronary and cerebral vascular thromboses)

of the patient (i.e. destruction of the CD4+ T-cell helper function). The development of resistance to HAART components has not proven to be a problem since selection pressure is removed by complete cessation of HAART.

The concept of immunization with the patient's own HIV during STI originated from the observation of the clinical course of the “Berlin patient” who was treated before complete treated with HAART early in the course of HIV infection in which CTL responses against gag antigens of HIV were preserved by introduction of HAART early in the course of infection. Suppression of plasma HIV RNA following STI was associated with strong CTL responses. STI in patients not treated early with HAART during HIV infection have demonstrated less successful suppression of HIV. Oritz et.al. report that two of six patients contained plasma virermia for twelve and twenty-four months, respectively, following STI of HAART. Strong CTL responses correlated with suppression of viremia. Similarly, Lori et al. using hydroxycarbamide modified HAART demonstrated an 180 day suppression of viremia in one of three patients. The difference in response rates between early HAART versus treatment started after complete seroconversion of Western blots would appear to relate to the preservation of CTL responses early in the course of HIV infection as compared to their absence once HIV infection enters its chronic phase. Potentiation of the CTL response during STI would, therefore, be a desirable goal for maximizing immune responses to control viremia and prolong HAART-free intervals since the expected relapse rate in just 30 days after stopping HAART is 86%.

### DESCRIPTION OF THE INVENTION

We have found that the administration of dsRNA at an appropriate stage in HAART therapy allows for the discontinuation of HAART by increasing the time to HIV rebound after stopping HAART. The dsRNA treatment leads to a reduced incidence of toxicity to antiretroviral therapy and reduces the overall costs associated with treating HIV infections. seroconversion of the Western blot response with a modified HAART regimen with a reduction of plasma HIV load from 85,000 copies/ml to undetectable. During a temporary suspension of HAART, viremia occurred transiently until resumption of HAART. During a second suspension of HAART, no HIV rebound occurred. The patient elected to stop HAART permanently after 176 days with no subsequent viral rebound during the following 551 days although traces of HIV RNA were detected in a lymph node and replication competent virus was isolated from resting CD4+ lymphocytes at very low frequencies. Thus, HIV in this patient had not been eradicated. Replication control was apparently provided by the cell mediated arm of the immune system since no neutralizing activity could be demonstrated and a strong CTL response to HIV p17 was observed. This observation in a single patient, nevertheless, supports the argument earlier (1997) suggesting increased focus on the cell-mediated arm of the immune system in order to control HIV infection. Recent studies confirm this insight and provide a rational mechanism for the role of STI in HAART.

A primary target for HIV is the CD4+ T-lymphocyte which accounts for its declining numbers during the course of HIV infection and the natural progression to AIDS. Although CD8+ T-cytolytic lymphocytes are not targets for HIV, their cytolytic capacity against infected cells presenting HIV epitopes is dependent on functional help from CD4+ cells. Thus, the CTL response is disarmed by an attack on CD4+ lymphocytes. With the loss of HIV memory cells during infection by HIV, chronic suppression of HIV by HAART provides no mechanism for the induction of specific CTL responses even with rising CD4+ levels. Rosenberg et.al. report the successful use of STI in five of eight patients who were

The invention includes methods of enhancing therapy against HIV by administering to patients whose HIV plasma RNA has been suppressed by active anti-retroviral therapy to a value below detection, typically less than 50 copies/ml, a synthetic, specifically configured, double-stranded ribonucleic acid (dsRNA) which retains the immunostimulatory and antiviral properties of other double stranded RNA molecules but exhibits greatly reduced toxicity. Concurrent anti-retroviral and dsRNA therapy is continued for a predetermined period of time, for example 2-4 months, then anti-retroviral therapy is discontinued while dsRNA therapy is maintained then, following an HIV rebound the HAART is restarted. A rebound may be determined by HIV plasma RNA of more than 5,000 copies/ml for three consecutive weeks or more than 50,000 copies/ml on a single occasion. While other indicators of HIV, presence/activity may be employed, such as change in CD4+ lymphocyte count, we prefer assessing MV plasma RNA as being both convenient and accurate based on the sensitive assay for same currently available.

The dsRNA of choice is Ampligen®, a synthetic, specifically configured, double-stranded ribonucleic acid (dsRNA) which retains the immunostimulatory and antiviral properties of other double-stranded RNA molecules (dsRNA) but exhibits greatly reduced toxicity. Like other dsRNA, Ampligen® can elicit the induction of interferon and other cytokines. Ampligen® has the ability to stimulate a variety of dsRNA-dependent intracellular antiviral defense mechanisms including the 2′,5′-oligoadenylate synthetase/RNase L and protein kinase enzyme pathways.

The mismatched dsRNA may be of the general formula rIₙ·r(C₁₂U)ₙ. In this and the other formulae that follow r=ribo. Other mismatched dsRNAs for use in the present invention are based on copolynucleotides selected from poly (Cₙ,U) and poly (CₙG) in which n is an integer having a value of from 4 to 29 and are mismatched analogs of complexes of polyriboinosinic and polyribocytidilic acids, formed by modifying rIₙ·rCₙ to incorporate unpaired bases (uracil or guanine) along the polyribocytidylate (rCₙ) strand. Alternatively, the dsRNA may be derived from r(I)·r(C) dsRNA by modifying the ribosyl backbone of polyriboinosinic acid (rIₙ), e.g., by including 2′-O-methyl ribosyl residues. The mismatched may be complexed with an RNA-stabilizing polymer such as lysine cellulose. Of these mismatched analogs of rIₙ·Cₙ, the preferred ones are of the general formula rIₙ·r(C₁₁₋₁₄,U)ₙ. or rIₙ·r(C₂₉,G)ₙ, and are described by Carter and Ts'o in U.S. Pat. Nos. 4,130,641 and 4,024,222 the disclosures of which are hereby incorporated by reference. The dsRNA's described therein generally are suitable for use according to the present invention.

Other examples of mismatched dsRNA for use in the invention include:
r(I)·r(C₄, U)
r(I)·r(C₇, U)
r(I)·r(C₁₃, U)
r(I)·r(C₂₂, U)
r(I)·r(C₂₀, G) and
r(I)·r(Cₚ₂₃,G_{>p}).

Alternatively the dsRNA may be the matched form, thus polyadenylic acid complexed with polyuridylic acid (poly A•poly U) may also be used. Clinical studies of Ampligen® have reported the following activities: decreases in viral load, stabilization of CD4 cell counts, and restoration of delayed type hypersensitivity (DTH) in anergic individuals infected with HIV. Despite the dramatic reduction of HIV load in patients on various highly active anti-retroviral therapy (HAART) regimens, the development of drug resistant mutants during therapy provides a significant challenge for long-term inhibition of HIV replication. The recent demonstration of synergy between Ampligen® and all three classes of currently FDA-approved drugs and the ability to inhibit drug-resistant mutants from each class has renewed interest in Ampligen® as a potential new drug with a new mechanism of action to inhibit HIV replication. Moreover, the immunomodulatory activity of Ampligen® suggests that the drug may function to reverse the Th1 to Th2 switch observed with HIV infection. Natural killer (NK) cell activity is also increased in Ampligen® treated nude mice bearing human bladder carcinoma, renal carcinoma and melanoma xenografts. Similarly, human PBMCs treated with Ampligen® respond with an increase in NK cell activity.

The following table lists the FDA approved antiretroviral drugs and drug combinations:

TABLE 2
Antiretroviral Drugs and Drug Combinations Approved by FDA for the
HIV Indication as of Dec. 31, 2001

| | |
|---|---|
| Abacavir (Ziagen) | Amprenavir (Agenerase) |
| Zidovudine (Retrovir) | Combivir |
| Zalcitabine (Hivid) | Lamivudine (Epivir) |
| Didanosine (Videx) | Trizivir |
| Stavudine (Zerit) | Lopinavir (Kaletra) |
| Efavirenz (Sustiva) | Nevirapine (Viramune) |
| Indinavir (Crixivan) | Delavirdine (Resciptor) |
| Ritonavir (Norvir) | Saquinavir (Fortovase or Invirase) |
| Nelfinavir (Viracept) | Tenofovir (Viread) |

The present invention includes the above combinations as well as other antiretroviral drugs and drug combinations yet to receive approval or acceptance in HAART.

Failure of antiretroviral therapies over time and the demonstration of resistance have stimulated intensive searches for appropriate combinations of agents, or sequential use of different agents, that act at the same or different viral targets. HAART is the utilization of several antiretrovirals with different mechanisms of actions to decrease viral loads in heavily experienced HIV-1 infected patients. This invention demonstrates the effectiveness of adding Ampligen® to HAART with regard to the duration of antiviral response, assessed by plasma HFV-1 RNA measurements (Roche Amplicor Assay) following a STI of HAART.

The use of dsRNAs as monotherapy in HIV disease is described in U.S. Pat. No. 4,820,696 and in combination with other anti-retroviral agents is described in U.S. Pat. No. 4,950,652.

Clinical Examples

An open-label, prospective, randomized, controlled study of the safety and biological effects, including clinical, immunologic, and virologic assessments, of adding Ampligen® 400 mg to a STI protocol of HAART containing at least one of the following ten antiretroviral drugs: Ziagen (abacavir), Retrovir (zidovudine) AZT, Hivid (zalcitabine) ddC, Videx (didanosine) ddI, Zerit (stavudine) d4T, Sustiva (efavirenz), Crixivan (indinavir), Norvir (ritonavir) Viracept (nelfinavir), and Agenerase (amprenavir), in patients with plasma HIV RNA <50 and CD4 levels ≧400.

Following Baseline evaluations (3 weeks) patients were stratified based on the presence of one versus the presence of two or more of the above-listed ten anti-retroviral drugs.

This study consisted of a period with a randomization (1:1/Ampligen®: No Ampligen®) into two parallel arms with 60 patients receiving Ampligen® and 60 receiving no Ampligen®. Poly I:poly C₁₂U (200 mg) was given by intravenous infusions (IV) twice weekly for four doses (Weeks 1 and 2) and then 400 mg IV twice weekly thereafter. The no Ampligen® arm received no IV infusions.

The primary study endpoint for efficacy is mean total time of the HAART-free intervals before rebound in plasma HIV-1 RNA (using the Roche Ultra Sensitive assay). A secondary efficacy endpoint is change in CD4+ lymphocyte count. Clinical status was followed. Safety and tolerance were determined by documentation and analysis of the number, type, relatedness, and severity of adverse events; by the reasons for early treatment discontinuation; and by any trends in clinical laboratory values indicating adverse effects.

All patients were on a HAART regimen that has suppressed HIV plasma RNA below the limits of detection (<50 copies/ml) during the last 9 months or longer. Following 8 weeks of Ampligen® or no Ampligen®, HAART was discontinued and patients were monitored weekly for HIV rebound (i.e.—HIV plasma RNA)>5000 copies/ml for 3 consecutive weeks or >50,000 on one occasion). Following HIV rebound, HAART is restarted. Eight (8) weeks after the plasma HIV RNA becomes undetectable, a second STI is introduced and monitored identically to the initial STI.

Thirty day STI data from six patients enrolled in this study were available. Three of these patients (coded S, W, and R in Table 3) were randomized to receive Ampligen® and three of these patients (coded J, M, and D in Table 4 below) were randomized to not receive Ampligen®.

As can be seen from Tables 3 and 4, all patients met the entrance criteria requiring a CD4 cell level >400, an HIV plasma RNA level <50 copies/ml, and a HAART regimen containing at least one anti-retroviral drug showing synergy with Ampligen® as listed above.

All patients were chronically HIV infected and were receiving the indicated HAART regimen prior to starting the STI. As shown in Table 4, during the first 30 days off of HAART, two of the three no Ampligen® patients relapsed with HIV plasma RNA levels increasing >1000 copies/ml compared to no relapses in the Ampligen® cohort (Table 3). In order to obtain a better estimate of the expected rate of relapse of this patient population when discontinuing HAART, a literature search and meta-analysis was utilized.

TABLE 3
Patient Characteristics AMP 720 Study (Ampligen ®)

| | | | CD4 Cell | | HAART¹ | |
|---|---|---|---|---|---|---|
| Patient | Age | | Count | HIV RNA | Received before | HIV |
| Code | (Years) | Risk Factor | (cells/mm³) | (copies/ml) | STI | Relapse² |
| S | 58 | Heterosexual | 400 | <50 | 3TC + ZDV + EFV | No |
| W | 64 | Homosexual | 540 | <50 | 3TC + ZDV + NVP | No |
| R | 44 | Heterosexual | 890 | <50 | 3TC + ZDV + NVP | No |

¹ABC, abacavir; SQV, saquinavir; NVP, nevirapine; LPV, lopinavir; NFV, nelfinavir; 3TC, lamivudine; ZDV, zidovudine; EFV, efavirenz.
²HIV Relapse = HIV RNA rebounded to ≧1000 copies/ml within first 30 days of discontinuing HAART

TABLE 4
Patient Characteristics AMP 720 Study (No Ampligen ®)

| | | | CD4 Cell | HIV RNA | | |
|---|---|---|---|---|---|---|
| Patient | Age | | Count | (copies/ | HAART¹ Received | HIV |
| Code | (Years) | Risk Factor | (cells/mm³) | ml) | before STI | Relapse² |
| J | 33 | Homosexual | 960 | <50 | 3TC + ZDV + EFV | Yes |
| M | 42 | Homosexual | 700 | <50 | ABC ÷ ZDV + 3TC + NFV | No |
| D | 51 | Homosexual | 530 | <50 | ABC ÷ LPV + SQV | Yes |

¹ABC, abacavir; SQV, saquinavir; NVP, nevirapine; LPV, lopinavir; NFV, nelfinavir; 3TC, lamivudine; ZDV, zidovudine; EFV, efavirenz.
²HIV Relapse = HIV RNA rebounded to ≧1000 copies/ml within first 30 days of discontinuing HAART

Meta-analysis is a quantitative approach for systematically combining the results of previous research and has become a popular technique in virtually every area of medicine. A search of the biomedical Literature was conducted to identify publications which contained data pertaining to the rate of HIV relapse during STIs of HAART in chronically infected HIV patients with CD4 cell levels ≧400 and HIV RNA plasma levels <50 prior to initiation of the STI. Two recent publications were identified which studied HIV relapse rates during the first 30 days following the start of the STI: Ruiz et al “HIV dynamics and T-cell immunity after three structured treatment interruptions in chronic HIV-1 infection” AIDS 2001, 15:F19-F27 and Birk et al “Kinetics of HIV-1 RNA and resistance-associated mutations after cessation of antiretroviral combination therapy” AIDS 2001, 15:1359-1368.

Study A, Ruiz et al, from the Hospital Universitari Germans Trias i Pujol, Badalona, Spain; Hôspital Pitié-Salpêtrière, Paris, France; and the Centre for HIV Research, Edinburgh, Scotland, UK examined HIV dynamics after structured treatment interruptions (STIs) in chronic HIV-1 infection. As shown in Table 5, all 12 patients had HIV plasma RNA levels <50 copies/ml, a CD4 level ≧400, and a HAART regimen containing at least one anti-retroviral drug showing synergy with Ampligen®. Ten of the 12 patients (all except patients 9 and 12) relapsed during the first 30 days off HAART with HIV plasma RNA increasing above 1000 copies/ml.

Study B, Birk et al, from the Karolinska Institute, Huddinge University Hospital, Stockholm, Sweden also examined the kinetics of HIV-1 RNA changes following the cessation of HAART. Of the 26 chronically infected patients studied, only nine of these patients had CD4 cell levels ≧400 and HIV plasma RNA levels <50 prior to start of the STI. These nine patients also had a HAART regimen containing at least one anti-retroviral drug showing synergy with Ampligen®. Data on these nine patients are shown in Table 6. Patient U was the only patient who did not relapse with IV plasma RNA increasing to ≧1000 copies/ml within the first 30 days after initiation of the STI.

The combined data from Studies A and B yield a relapse rate of 86% (18/21) within the first 30 days of stopping HAART in chronically infected HIV patients.

A meta-analysis combining the data from studies A and B with the interim results of AMP 720 is shown in Table 7.

TABLE 5
Patient Characteristics Study A¹

| Patient | | | CD4 Cell Count | HIV RNA | HAART² Received | |
|---|---|---|---|---|---|---|
| Code | Age (Years) | Risk Factor⁴ | (cells/mm³) | (copies/ml) | before STI | HIV Relapse³ |
| 1 | 33 | IVDU | 2870 | <50 | 3TC + d4T + IDV | Yes |
| 2 | 37 | IVDU | 742 | <50 | ZDV + ddI + IDV + HU | Yes |
| 3 | 29 | IVDU | 1673 | <50 | 3TC + d4T + IDV | Yes |
| 4 | 32 | Heterosexual | 1141 | <50 | 3TC + d4T + NFV | Yes |
| 5 | 41 | IVDU | 1189 | <50 | 3TC + d4T + NFV | Yes |
| 6 | 39 | Heterosexual | 828 | <50 | 3TC + d4T + SQV | Yes |
| 7 | 28 | Heterosexual | 1311 | <50 | 3TC + d4T + IDV | Yes |
| 8 | 29 | Heterosexual | 1837 | <50 | 3TC + d4T + IDV | Yes |
| 9 | 27 | Heterosexual | 1349 | <50 | 3TC + d4T + IDV | No |
| 10 | 38 | Homosexual | 1486 | <50 | 3TC + d4T + IDV | Yes |
| 11 | 38 | Homosexual | 1002 | <50 | 3TC + d4T + IDV | Yes |
| 12 | 42 | IVDU | 979 | <50 | 3TC + d4T + RTV | No |

¹AIDS 15: F19-F27 (2001)
²d4T, stavudine; ddI, didanosine; HU, hydroxyurea; IDV, indinavir; NFV, nelfinavir; RTV, ritonavir; SQV, saquinavir; 3TC, lamivudine; ZDV, zidovudine.
³HIV Relapse = HIV RNA rebounded to ≧1000 copies/ml within first 30 days of discontinuing HAART
⁴IVDU, intravenous drug user.

TABLE 6
Patient Characteristics Study B¹

| | | | CD4 Cell Count | HIV RNA | HAART² Received | |
|---|---|---|---|---|---|---|
| Patient Code | Age (Years) | Risk Factor⁴ | (cells/mm³) | (copies/ml) | before STI | HIV Relapse³ |
| D | 32 | Homosexual | 500 | <50 | ddI, EFV, d4T | Yes |
| F | 42 | IVDU | 550 | <50 | 3TC, d4T, NFV | Yes |
| L | 34 | Heterosexual | 580 | <50 | d4T, 3TC, NFV | Yes |
| M | 25 | Heterosexual | 760 | <50 | ZDV, 3TC, ddI | Yes |
| Q | 61 | Homosexual | 710 | <50 | d4T, ddI, RTV, IDV | Yes |
| T | 56 | IVDU | 400 | <50 | d4T, 3TC, NFV | Yes |
| U | 43 | Heterosexual | 1410 | <50 | ZDV, 3TC, EFV | No |
| V | 42 | Homosexual | 500 | <50 | d4T, 3TC, IDV, RTV | Yes |
| Y | 41 | Heterosexual | 520 | <50 | d4T, 3TC, NFV | Yes |

¹AIDS 15: 1359-1368 (2001)
²d4T, stavudine; ddI, didanosine; IDV, indinavir; NFV, nelfinavir; RTV, ritonavir; 3TC, lamivudine; ZDV, zidovudine; EFV, efavirenz.
³HIV Relapse = HIV RNA rebounded to ≧1000 copies/ml within first 30 days of discontinuing HAART
⁴IVDU, intravenous drug user

The meta-analysis (Table 7) shows that the 0% relapse rate for the Ampligen® cohort following the STI of HAART is significantly lower (p=0.012) than expected for this chronically infected population.

TABLE 7
Meta-Analysis of AMP 720 Interim Results Showing a
Decreased HIV Relapse Rate with Ampligen ® Treatment

| | No Relapses - | Relapses - | |
|---|---|---|---|
| Treatment | # Patients (%) | # Patients (%) | p-value* |
| Ampligen ® | 3 (100%) | 0 (0%) | 0.012 |
| No Ampligen ® | 4 (16.7%) | 20 (83.3%) | |

*Fisher's Exact Test

A safety analysis summarized in the attached Table 8 shows no evidence of increased toxicity. Blood laboratory studies at Week 8 were compared to Baseline values for the Ampligen® and no Ampligen® cohorts. As can be seen in Table 8 there was no evidence of any added toxicity to the bone marrow, kidneys, or liver by the addition of Ampligen® to the patient's HAART regimen. Thus, these data suggest that the clinical benefit of Ampligen® treatment can be obtained without any significant additional toxicity.

TABLE 8
Ampligen ® Plus HAART Shows No Evidence of Toxicity

| | | | Ampligen | Mean | Mean | Mean Change | |
|---|---|---|---|---|---|---|---|
| Parameter | Normal Ranges | | Treatment | BSL* | Week 8 | Week 8 - BSL | p-value⁺ |
| Hemoglobin | 12.5-17.0 | g/dL | YES | 13.5 | 13.1 | −0.4 | 0.946 |
| | | | NO | 14.5 | 14.0 | −0.4 | |
| White Blood Count | 4.0-10.5 × 10⁻³ | /uL | YES | 5.5 | 4.9 | −0.6 | 0.726 |
| | | | NO | 7.2 | 6.1 | −1.1 | |
| Platelet Count | 140-415 × 10⁻³ | /uL | YES | 271.4 | 241.0 | −30.4 | 0.102 |
| | | | NO | 281.7 | 281.7 | 0.0 | |
| Creatinine | 0.5-1.5 | mg/dL | YES | 0.8 | 0.7 | −0.1 | 0.270 |
| | | | NO | 1.1 | 1.0 | 0.0 | |
| BUN | 5-26 | mg/dL | YES | 14.5 | 13.7 | −0.8 | 0.342 |
| | | | NO | 14.0 | 15.7 | 1.7 | |
| Gamma-GT | 0-65 | IU/L | YES | 64.8 | 74.3 | 9.5 | 0.758 |
| | | | NO | 58.3 | 77.0 | 18.7 | |
| SGPT(ALT) | 0-40 | IU/L | YES | 46.0 | 42.7 | −3.3 | 0.304 |
| | | | NO | 37.5 | 45.7 | 8.2 | |
| Alkaline | 25-150 | IU/L | YES | 105.7 | 89.0 | −16.7 | 0.316 |
| Phosphatase | | | NO | 109.8 | 102.3 | −7.5 | |
| Bilirubin, Total | 0.1-1.2 | mg/dL | YES | 0.4 | 0.3 | −0.1 | 0.529 |
| | | | NO | 0.5 | 0.5 | 0.0 | |

*BSL = Baseline + t-test (two-sided)

1. A method of mitigating the adverse effects of antiviral agents in HIV therapy comprising administering to an HIV-infected subject at least one antiviral agent until HIV is suppressed, discontinuing antiviral therapy, administering a dsRNA, then, when HIV values increase resuming antiviral treatment with the at least one antiviral agent.

2. he method of claim 1 wherein step a dsRNA is administered with said antiviral agent.

3. The method of claim 1 or wherein step HIV values are suppressed to a value below detection.

4. The method of claim 3 wherein the value is less than 50 copies/ml of HIV plasma RNA.

5. The claim 1 wherein two or more anti-retroviral agents are used.

6. The method of claim 5 wherein the anti-retroviral drug is selected from abacavir, amprenavir, zidovudine, combivir, zalcitabine, lamivudine, didanosine, trizivir, stavudine, lopinavir, efavirenz, nevirapine, indinavir, delavirdine, ritonavir, saquinavir, nelfinvir and tenofovir.

7. The method of claim 1 where the dsRNA is rIₙ r(C₁₂U)ₙ, Poly A Poly U or rI_{n r(C}₂₉,G)ₙ, in which r is ribo and n has a value of 4 to 29.
